(19) United States
(12) Patent Application Publication
(10) Pub. No.: US 2013/0259960 A1
(43) Pub. Date: Oct. 3, 2013

(54) TOPICAL TREATMENT FOR MENOPAUSAL HOT FLASHES

(71) Applicants: Frances Anne Wiesener, Carrabelle, FL (US); Shelley Kay McLean, Lanark Village, FL (US)

(72) Inventors: Frances Anne Wiesener, Carrabelle, FL (US); Shelley Kay McLean, Lanark Village, FL (US)

(73) Assignee: Frances Anne Wiesener, Carrabelle, FL (US)

(21) Appl. No.: 13/609,507

(22) Filed: Sep. 11, 2012

Related U.S. Application Data
(60) Provisional application No. 61/618,875, filed on Apr. 2, 2012.

(51) Int. Cl. A61K 36/61; A61P 15/00

(52) U.S. Cl. 424/742

(57) ABSTRACT

A topical treatment for menopausal hot flashes including essential oils of Clove, Eucalyptus, and Peppermint, carried in a carrier oil.

### CROSS-REFERENCE TO RELATED APPLICATIONS

This application claims priority under 35 USC 119 to U.S. Provisional Application No. 61/618,875 filed Apr. 2, 2012, which is incorporated by reference herein in its entirety.

### BACKGROUND

Hot flashes (aka hot flushes) are the most frequent symptoms of menopause and perimenopause. Hot flashes occur in more than two-thirds of North American women during perimenopause and almost all women with induced menopause or premature menopause.

A hot flash—sometimes called a hot flush—is a momentary sensation of heat that may be accompanied by a red, flushed face and sweating. The cause of hot flashes is not known, but may be related to changes in circulation.

Hot flashes occur when the blood vessels near the skin's surface dilate to cool. This produces the red, flushed look to the face. A woman may also perspire to cool down the body. In addition, some women experience a rapid heart rate or chills.

Hot flashes accompanied with sweating can also occur at night. These can be called night sweats and may interfere with sleep.

A hot flush can be a hot flash plus a visual appearance of redness in the face and neck.

The severity and duration of hot flashes varies among women going through menopause. Some women have hot flashes for a very short time during menopause. Other women may have hot flashes—at least to some degree—for life. Generally, hot flashes are less severe as time passes.

In the prior art it has be said that while it may be impossible to completely avoid hot flashes during menopause, there are certain triggers that may bring them on more frequently or cause them to be more severe. To prevent hot flashes, prior literature has noted to avoid triggers such as stress, caffeine, spicy foods, alcohol, and tight clothing.

Other things the prior art has suggested to keep hot flashes at bay include trying to keep the body cool at night, breathing control at the onset of a hot flash, etc.

The prior art has also includes the use of hormone replacement therapy, or HRT. This treatment can reduce hot flashes in many women and can help other symptoms of menopause, including vaginal dryness and mood disorders. However, even short-term hormone therapy carries some risks, including blood clots and gallbladder inflammation.

Other prior art treatments have been found that may offer relief. These include both over-the-counter and prescription therapies. Nonprescription treatments include Vitamin B complex, Vitamin E and Ibuprofen.

Prescription treatments include Catapres, Catapres-TTS, Aldomet, blood pressure medications, birth control pills, antidepressants (such as Zoloft, Paxil, and Effexor) other hormones (such as Provera and Megace), and Neurontin, an anti-seizure drug.

Alternative therapies like botanical and herbal therapies have been used in the prior art for relieving menopausal symptoms such as hot flashes, although their success is debated.

Therefore it may be seen that there is a need in the art for an improved treatment of menopausal hot flashes.

### BRIEF SUMMARY OF THE INVENTION

Generally described, the present invention relates to a topical treatment for menopausal hot flashes. Described in further detail, the invention relates to the provision of a composition for topical use on a human to reduce menopausal hot flashes. When used as directed, the severity and frequency of menopausal hot flashes (flushes) is reduced.

One embodiment of the present invention includes a composition for topical application in the treatment of menopausal related discomfort, said mixture comprising: A) approximately 8% by volume of essential oil of Clove; B) approximately 8% by volume of essential oil of Eucalyptus; C) approximately 8% by volume of essential oil of Peppermint; and D) approximately 76% by volume of carrier oil. Under a variation of this embodiment, the carrier oil could be grape seed carrier oil.

Another embodiment of the present invention includes a composition for topical application in the treatment of menopausal related discomfort, said mixture comprising: A) approximately 1 part by volume of essential oil of Clove; B) approximately 1 part by volume of essential oil of Eucalyptus; C) approximately 1 part by volume of essential oil of Peppermint; and D) approximately 12 parts by volume of carrier oil. Under a variation of this embodiment, the carrier oil could be grape seed carrier oil.

Another embodiment of the present invention includes a composition for topical application in the treatment of menopausal related discomfort, said mixture comprising: A) approximately 1 part by volume of essential oil of Clove; B) approximately 1 part by volume of essential oil of Eucalyptus; C) approximately 1 part by volume of essential oil of Peppermint; and D) a therapeutically effective amount of carrier oil. Under a variation of this embodiment, the carrier oil could be grape seed carrier oil. Under another variation of this embodiment, the carrier oil could be in the range of 4-27 parts.

Another embodiment of the present invention includes a composition for topical application in the treatment of menopausal related discomfort, said mixture comprising: A) approximately 3-20% by volume of essential oil of Clove; B) approximately 3-20% by volume of essential oil of Eucalyptus; C) approximately 3-20% by volume of essential oil of Peppermint; and D) approximately 40-91% by volume of carrier oil. Under a variation of this embodiment, the carrier oil could be grape seed carrier oil. Under another variation of this embodiment, the carrier oil could be in the range of 4-27 parts.

Another embodiment of the present invention includes a composition for topical application in the treatment of menopausal related discomfort, said mixture comprising: A) approximately 5-15% by volume of essential oil of Clove; B) approximately 5-15% by volume of essential oil of Eucalyptus; C) approximately 5-15% by volume of essential oil of Peppermint; and D) approximately 55-85% by volume of carrier oil. Under a variation of this embodiment, the carrier oil could be grape seed carrier oil. Under another variation of this embodiment, the carrier oil could be in the range of 4-27 parts.

Another embodiment of the present invention includes a composition for topical application in the treatment of menopausal related discomfort, said mixture comprising: A) approximately 8-10% by volume of essential oil of Clove; B) approximately 8-10% by volume of essential oil of Eucalyptus; C) approximately 8-10% by volume of essential oil of Peppermint; and D) approximately 70-76% by volume of carrier oil. Under a variation of this embodiment, the carrier oil could be grape seed carrier oil. Under another variation of this embodiment, the carrier oil could be in the range of 4-27 parts.

It is a further embodiment of the present invention to apply said mixtures above topically to the skin of said human in the amount of from one teaspoon once daily to one teaspoon twice daily.

Therefore an aspect of the present invention is to provide a treatment for hot flashes.

It is another aspect of the present invention to provide a treatment for hot flashes which is effective.

It is another aspect of the present invention to provide a treatment for hot flashes which is simple.

It is another aspect of the present invention to provide a treatment for hot flashes which is cost-effective.

Other aspects and inventions referenced in the originally filed claims of this application are also included.

It is to be understood that both the foregoing general description and the following detailed description are exemplary and explanatory and are intended to provide further explanation of the invention as claimed.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a drawing showing a human applicator 5 manually applying product 10 (via hands 6) to the skin 8 of a recipient 7.

FIG. 2 shows self-application of the product to a user's leg.

FIG. 3 shows self-application of the product to a user's arm proximate the shoulder.

FIG. 4 shows a chart including an exemplary component mixture.

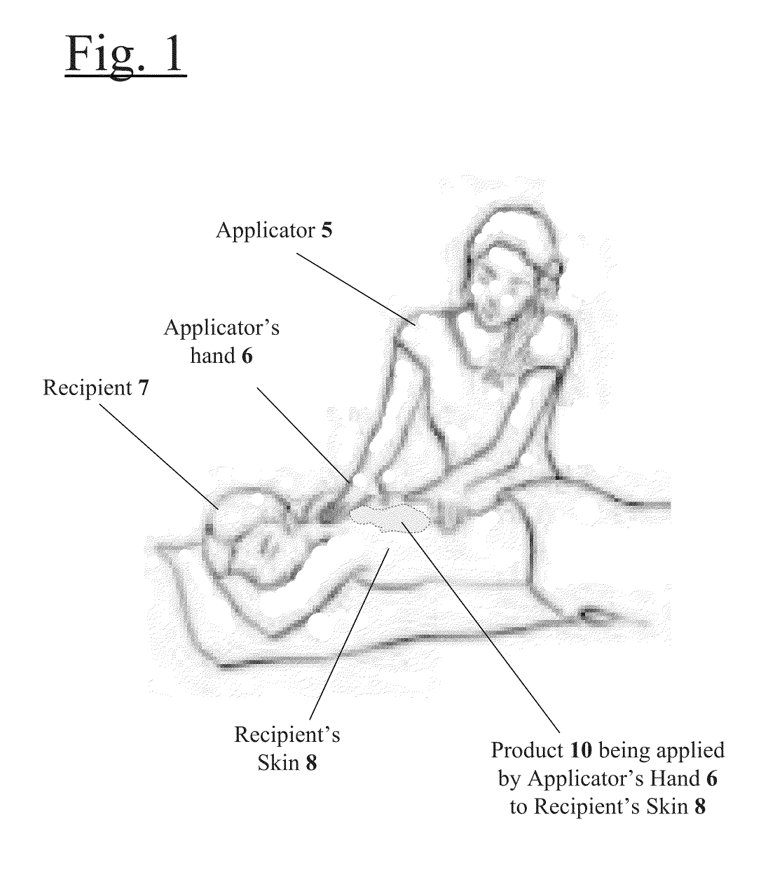

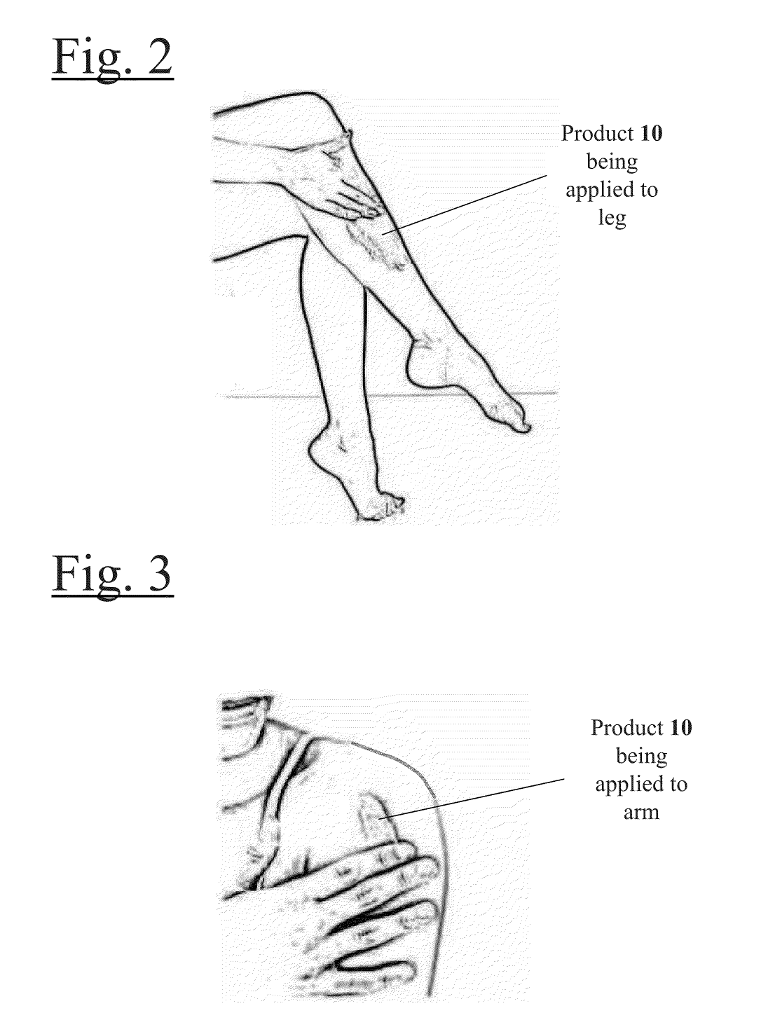

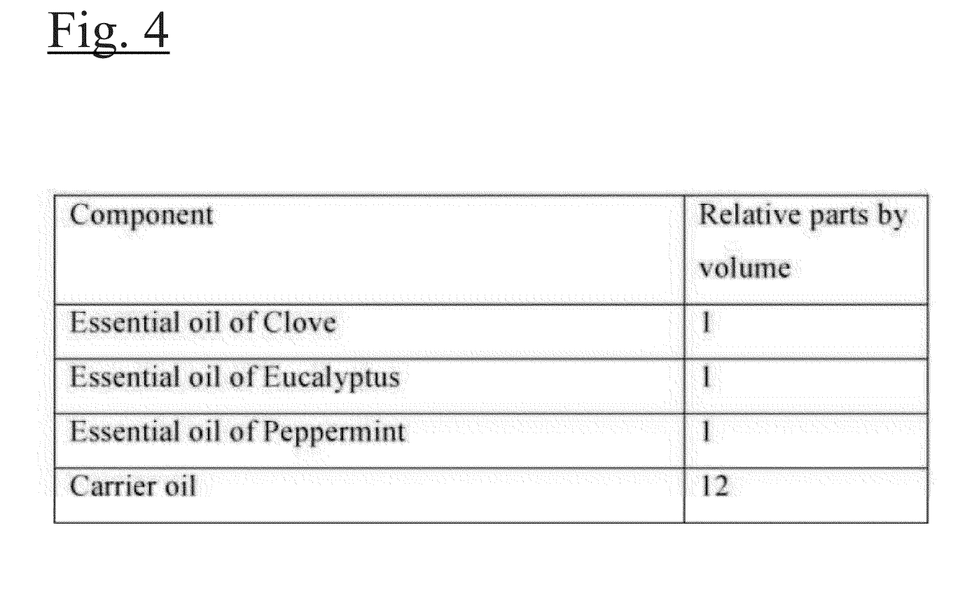

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

General Summary

Generally described, the present invention relates to a topical treatment for menopausal hot flashes. When used as directed, the severity and frequency of menopausal hot flashes (flushes) is reduced.

Various mixtures, and ranges of mixtures are contemplated under the present invention. Various application protocols are also contemplated under the present invention.

Element List

5 Applicator

6 Applicator's hand

7 Recipient

8 Recipient's skin

10 Product

20 Chart

Detailed Discussion

The Product Generally

The invention is made by combining the three essential oils and the carrier oil in a specific ratio. The product is then applied topically to the skin.

When used as directed, the severity and frequency of menopausal hot flashes (flushes) is reduced.

Carrier Oils

A carrier oil, also known as fixed or base oil, is usually a vegetable oil although in massage therapy there are uses of animal oils such as emu or fish oil. Animal oils however are not typically used in aromatherapy.

Carrier oils are used to dilute essential oils and absolutes before they are applied to the skin in massage and aromatherapy. They are so named because they carry the essential oil onto the skin. Carrier oils do not typically contain a concentrated aroma, unlike essential oils, though some, such as olive, have a mild distinctive smell. Neither do they evaporate like essential oils, which tend to be more volatile. The carrier oils used should be as natural and unadulterated as possible. Organic oils should be used where possible. Cold-pressing and maceration are the two main methods of producing carrier oils.

For purposes of the present discussion, one type of carrier oil could be grape seed carrier oil, but any suitable carrier oil could be used. Mineral oil should never be used as a carrier oil.

Essential Oils

An essential oil is a concentrated hydrophobic liquid containing volatile aroma compounds from plants. Essential oils are also known as volatile oils, ethereal oils or aetherolea, or simply as the “oil of” the plant from which they were extracted, such as oil of clove. An oil is “essential” in the sense that it carries a distinctive scent, or essence, of the plant.

Essential oils are unique to other oils in carrying the “essence” of the plant from which they are extracted. Essential oils are considered to be 100% pure. There are, however, some variations in concentration of the active ingredient, depending on the part of the plant from which the essence is extracted or from the species of the plant use. Essential oils should never be applied without dilution as they can be harmful to the skin and are volatile preventing absorption into the body, hence the need for carrier oil.

The particular essential oils used in the present application are now discussed.

In the case of clove oil, the active ingredient is eugenol. Clove oil extracted from the flower bud contains 60-90% eugenol; from the leaf contains 82-88% eugenol; and from the stem contains 90-95% eugenol. Pennyroyal from the leaf is most commonly, but not exclusively used. Pennyroyal is the clove oil used in the formulas, but one could use any clove oil if different concentrations are accounted for. Medline Plus reports that use of clove oil on the skin seems to be safe. It is not safe for oral use, inhalation or intravenous use.

Peppermint (mentha piperita) essential oil is the most commonly available and used peppermint essential oil, and is the peppermint oil used in the formulas, but one could use any peppermint oil if different concentrations are accounted for. Another variety, mentha arvensis, is made and sold, but is reported not to have the same useful medicinal properties as peppermint piperita. Peppermint oil is reported safe for topical application in a dilution of 5-20% solution.

Eucalyptus oils in the trade are categorized into three broad types according to their composition and main end-use: medicinal, perfumery and industrial. The most prevalent is the standard cineole based oil of eucalyptus. Global production is dominated by Eucalyptus globulus, which is what is used in the mixtures discussed herein. However, Eucalyptus kochii and Eucalyptus polybractea have the highest cineole content, ranging from 80-95%. The British Pharmacopoecia states that the oil must have a minimum cineole content of 70% if it is pharmaceutical grade. Recification is used to bring the lower grades up to the high cineole standard required.

Various Mixtures

Here follows various mixtures contemplated under the present invention. For purposes of this discussion, one “part” by volume could be any consistently used unit of volume, including but not limited to the milliliter.

One Preferred Formula is:

| | Relative parts by |
|---|---|
| Component | volume |
| Essential oil of Clove | 1 |
| Essential oil of Eucalyptus | 1 |
| Essential oil of Peppermint | 1 |
| Carrier oil | 12 |

Another Preferred Formula is:

| | Percentage by |
|---|---|
| Parts | volume |
| Essential oil of Clove | 8% |
| Essential oil of Eucalyptus | 8% |
| Essential oil of Peppermint | 8% |
| Carrier oil | 76% |

Another Preferred Maximum Formula is:

| | Relative parts by | Percentage by |
|---|---|---|
| Parts | volume | volume |
| Essential oil of Clove | 1 | 20% |
| Essential oil of Eucalyptus | 1 | 20% |
| Essential oil of Peppermint | 1 | 20% |
| Carrier oil | 4 | 40% |

Another Preferred Minimum Formula is:

| | Relative parts by |
|---|---|
| Parts | volume |
| Essential oil of Clove | 1 |
| Essential oil of Eucalyptus | 1 |
| Essential oil of Peppermint | 1 |
| Carrier oil | 27 |

Another Preferred Max Range of Carrier Formula is:

| | Relative parts by |
|---|---|
| Parts | volume |
| Essential oil of Clove | 1 |
| Essential oil of Eucalyptus | 1 |
| Essential oil of Peppermint | 1 |
| Carrier oil | 4-27 |

Another Preferred Max Range Formula is:

| | Percentage by |
|---|---|
| Parts | volume |
| Essential oil of Clove | 3-20% |
| Essential oil of Eucalyptus | 3-20% |
| Essential oil of Peppermint | 3-20% |
| Carrier oil | 40-91% |

Another Preferred Intermediate Range Formula (“Range 2”) is:

| | Percentage by |
|---|---|
| Parts | volume |
| Essential oil of Clove | 5-15% |
| Essential oil of Eucalyptus | 5-15% |
| Essential oil of Peppermint | 5-15% |
| Carrier oil | 55-85% |

Another Preferred Intermediate Range Formula (“Range 3”) is:

| | Percentage by |
|---|---|
| Parts | volume |
| Essential oil of Clove | 7-10% |
| Essential oil of Eucalyptus | 7-10% |
| Essential oil of Peppermint | 7-10% |
| Carrier oil | 70-79% |

Another Preferred Minimum Formula is:

| | Percentage by |
|---|---|
| Parts | volume |
| Essential oil of Clove | 3% |
| Essential oil of Eucalyptus | 3% |
| Essential oil of Peppermint | 3% |
| Carrier oil | 91% |

Another Preferred Intermediate Formula is:

| | Percentage by |
|---|---|
| Parts | volume |
| Essential oil of Clove | 7% |
| Essential oil of Eucalyptus | 7% |
| Essential oil of Peppermint | 7% |
| Carrier oil | 79% |

Another Preferred Intermediate Formula is:

| | Percentage by |
|---|---|
| Parts | volume |
| Essential oil of Clove | 10% |
| Essential oil of Eucalyptus | 10% |
| Essential oil of Peppermint | 10% |
| Carrier oil | 70% |

Another Preferred Intermediate Formula is:

| | Percentage by |
|---|---|
| Parts | volume |
| Essential oil of Clove | 15% |
| Essential oil of Eucalyptus | 15% |
| Essential oil of Peppermint | 15% |
| Carrier oil | 55% |

Application of the Product

The invention is used by applying the mixture to the skin in specific areas and at specific intervals of time.

Under one embodiment, an application method is to apply 1 teaspoon once daily to arms, legs and/or back, and to continue massaging oil until it is absorbed into the skin.

Under another embodiment, an application method is to apply 1 teaspoon twice daily to arms, legs and/or back, and to continue massaging oil until it is absorbed into the skin.

Additional Comments Regarding Mixture Concentrate

The inventors have found that if too little concentration of essential oils is used, then the effectiveness of the resulting mixture is correspondingly diluted. However, if too little carrier oil is used, the subject user can become uncomfortably “chilled” for an extended period of time. In one case, a user was chilled for approximately 2 (two) days.

Conclusion

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A composition for topical application in the treatment of menopausal related discomfort, said mixture comprising:
A) approximately 8% by volume of essential oil of Clove;
B) approximately 8% by volume of essential oil of Eucalyptus;
C) approximately 8% by volume of essential oil of Peppermint; and
D) approximately 76% by volume of carrier oil.

2. The composition as claimed in claim 1, wherein said carrier oil is grape seed carrier oil.

3. A composition for topical application in the treatment of menopausal related discomfort, said mixture comprising:
A) approximately 1 part by volume of essential oil of Clove;
B) approximately 1 part by volume of essential oil of Eucalyptus;
C) approximately 1 part by volume of essential oil of Peppermint; and
D) approximately 12 parts by volume of carrier oil.

4. The composition as claimed in claim 1, wherein said carrier oil is grape seed carrier oil.

5. A composition for topical application in the treatment of menopausal related discomfort, said mixture comprising:
A) approximately 1 part by volume of essential oil of Clove;
B) approximately 1 part by volume of essential oil of Eucalyptus;
C) approximately 1 part by volume of essential oil of Peppermint; and
D) a therapeutically effective amount of carrier oil.

6. The composition as claimed in claim 5, wherein said carrier oil is grape seed carrier oil.

7. The composition as claimed in claim 6, wherein said therapeutically effective amount of carrier oil is in the range of 4-27 parts.

8. The composition as claimed in claim 5, wherein said therapeutically effective amount of carrier oil is in the range of 4-27 parts.

9. A composition for topical application in the treatment of menopausal related discomfort, said mixture comprising:
A) approximately 3-20% by volume of essential oil of Clove;
B) approximately 3-20% by volume of essential oil of Eucalyptus;
C) approximately 3-20% by volume of essential oil of Peppermint; and
D) approximately 40-91% by volume of carrier oil.

10. The composition as claimed in claim 9, wherein said carrier oil is grape seed carrier oil.

11. A composition for topical application in the treatment of menopausal related discomfort, said mixture comprising:
A) approximately 5-15% by volume of essential oil of Clove;
B) approximately 5-15% by volume of essential oil of Eucalyptus;
C) approximately 5-15% by volume of essential oil of Peppermint; and
D) approximately 55-85% by volume of carrier oil.

12. The composition as claimed in claim 9, wherein said carrier oil is grape seed carrier oil.

13. A composition for topical application in the treatment of menopausal related discomfort, said mixture comprising:
A) approximately 8-10% by volume of essential oil of Clove;
B) approximately 8-10% by volume of essential oil of Eucalyptus;
C) approximately 8-10% by volume of essential oil of Peppermint;
D) approximately 70-76% by volume of carrier oil.

14. The composition as claimed in claim 9, wherein said carrier oil is grape seed carrier oil.

15. A method for treating menopausal related discomfort of a human having skin, said method comprising the steps of:
A) providing a mixture comprising a therapeutically effective amount of:
(i) essential oil of Clove;
(ii) essential oil of Eucalyptus;
(iii) essential oil of Peppermint; and
(iv) a carrier oil; and
B) applying said mixture topically to the skin of said human.

16. The method as claimed in claim 15, wherein step “A” includes the use of grape seed carrier oil for the oil in subsection (iv).

17. The method as claimed in claim 15, wherein step “A” includes the step of providing a mixture comprising a therapeutically effective amount of:
i) approximately 8% by volume of essential oil of Clove;
ii) approximately 8% by volume of essential oil of Eucalyptus;
iii) approximately 8% by volume of essential oil of Peppermint; and
iv) approximately 76% by volume of carrier oil;

18. The method for treating menopausal related discomfort of a human having skin as claimed in claim 17, wherein step “B” comprises applying said mixture topically to the skin of said human in the amount of from one teaspoon once daily to one teaspoon twice daily.

19. The method as claimed in claim 15, wherein step “A” includes the step of providing a mixture comprising a therapeutically effective amount of:
i) approximately 1 part by volume of essential oil of Clove;
ii) approximately 1 part by volume of essential oil of Eucalyptus;
iii) approximately 1 part by volume of essential oil of Peppermint; and
iv) approximately 12 parts by volume of carrier oil;

20. The method for treating menopausal related discomfort of a human having skin as claimed in claim 19, wherein step “B” comprises applying said mixture topically to the skin of said human in the amount of from one teaspoon once daily to one teaspoon twice daily.

21. A method for treating menopausal related discomfort of a human having skin, said method comprising the steps of:
A) providing a mixture comprising a therapeutically effective amount of:
(i) approximately 3-20% by volume of essential oil of Clove;
(ii) approximately 3-20% by volume of essential oil of Eucalyptus;
(iii) approximately 3-20% by volume of essential oil of Peppermint;; and
(iv) approximately 40-91% by volume of carrier oil; and
B) applying said mixture topically to the skin of said human.

22. The method as claimed in claim 21, wherein step “A” includes the step of providing a mixture comprising a therapeutically effective amount of:
i) approximately 5-15% by volume of essential oil of Clove;
ii) approx approximately 5-15% by volume of essential oil of Eucalyptus;
iii) approximately 5-15% by volume of essential oil of Peppermint; and
iv) approximately 55-85% by volume of carrier oil;

23. The method as claimed in claim 21, wherein step “A” includes the step of providing a mixture comprising a therapeutically effective amount of:
i) approximately 7-10% by volume of essential oil of Clove;
ii) approx approximately 7-10% by volume of essential oil of Eucalyptus;
iii) approximately 7-10% by volume of essential oil of Peppermint; and
iv) approximately 70-79% by volume of carrier oil;
